(19) United States
(12) Patent Application Publication
(10) Pub. No.: US 2004/0152194 A1
(43) Pub. Date: Aug. 5, 2004

(54) Method of promoting the take of nitrogen fixing microorganisms to plants by making use od radiation treatment

(75) Inventors: Tamikazu Kume, Takasaki-shi (JP); Hidefumi Takeshita, Naka-gun (JP); Shu Fujimaki, Takasaki-shi (JP); Takuji Ohyama, Niigata-shi (JP); Norikuni Ohtake, Niigata-shi (JP)

Correspondence Address: BANNER & WITCOFF, 1001 G STREET N W, SUITE 1100, WASHINGTON, DC, 20001, US

(73) Assignee: Japan Atomic Energy Research Institute, Kashiwa-shi (JP)

(21) Appl. No.: 10/760,473

(22) Filed: Jan. 21, 2004

(30) Foreign Application Priority Data
Jan. 21, 2003 (JP) 12057/2003

(51) Int. Cl. A01N 63/00; C12N 15/01

(52) U.S. Cl. 435/446; 504/117

(57) ABSTRACT

A method of enhancing the take of a nitrogen fixing microorganism by exposing it to low-energy electron beams, wherein the energy of electron beams is so controlled that an adequate dose for enhancing the take of the nitrogen fixing microorganism on the root of a plant is applied to the desired site on the epidermis of the plant's tissue.

### BACKGROUND OF THE INVENTION

This invention provides a method in which the seeds or bodies of leguminous and various other plants are exposed to the required doses of electron beams with the depth of exposure being controlled by changing their energy so that the take of nitrogen fixing microorganisms on plant roots is enhanced without causing damage to the plants.

Among soil microorganisms are those which offer direct help to plant growth, as exemplified by root nodule bacteria which, in symbiosis with soybean or other leguminous plants, form organs called root nodules on their plants, fix atmospheric nitrogen and supply it to the plants, mycorrhiza fungi which, in symbiosis with the roots of many plants, extend hyphae to a remote site, thereby enlarging the nutrient absorbing area sufficiently to assist in phosphorus absorption, as well as bacteria that promote the lysis of nutrients such as phosphorus and iron that are not readily utilized by plants. Biofertilizers which depend on those microorganisms for crop production can increase the crop yield while reducing the use of chemical fertilizers and, hence, they can meet both requirements for crop production and environmental preservation. Particularly effective is the method of enhancing the take of nitrogen fixing microorganisms on plant roots since it can increase crop production without causing adverse effects on the environment.

Of particular interest in this regard is leguminous plants such as soybean which produce swellings called “root nodules” on their roots, keep root nodule bacteria as a soil microorganism, convert atmospheric nitrogen gas into ammonia which they use as a nutrient. The process of converting atmospheric nitrogen gas into ammonia is generally referred to as nitrogen fixation.

It has been known since ancient times that cultivation of beans makes the soil fertile and in rotational agriculture in Europe, leguminous grasses such as clover are planted instead of fallowing with a view to improving soil fertility. Microorganisms that perform nitrogen fixation are limited to prokaryotes free of cell nucleus such as bacteria, actinomycetes and blue-green algae, among which are microorganisms that perform nitrogen fixation in symbiosis with plants. The most famous example of symbiotic nitrogen fixation is the relationship between bean and root nodule bacterium. In addition to legumes, pioneer plants which are the first to emerge into wastelands, such as Alnus japonica Steudel, Myrica rubra S. et Z. and Casuarina stricta Ait., perform nitrogen fixation in the root nodules they form in symbiosis with actinomycetes. Cycas revoluta Thunb. forms root nodules that are symbiotic with blue-green algae. Aside from root nodule bacteria, certain microorganisms called endophytes which live around roots or within roots or stems can fix nitrogen and of particular interest is nitrogen fixation by sugar cane endophytes which grow vigorously despite poor nutrition.

However, those microorganisms take only poorly on plants and current practice is to apply biofertilizers that contain large amounts of artificially cultured effective cells. A need therefore exists to establish a more convenient and efficient method of achieving good take of nitrogen fixing microorganisms.

### SUMMARY OF THE INVENTION

The present invention has been accomplished under the circumstances and has as an object providing a method by which the take of nitrogen fixing microorganisms on the roots of plants is enhanced without impairing the inherent function of the plants so that they can acquire more nutrition as through nitrogen fixation, whereby the use of chemical fertilizers is reduced to help preserve the environment while permitting efficient plant growth.

The present inventors conducted intensive studies with a view to attaining the stated object and discovered a technique of employing low-energy electron beams for controlling the depth of exposure such that the right dose of electron beams are applied to cell walls on seed coats or the surfaces of plant bodies without impairing the inherent function of the plants. Based on this technique, the inventors successfully developed a method of enhancing the take of nitrogen fixing microorganisms on plant roots.

### BRIEF DESCRIPTION OF THE DRAWINGS

FIG. 1 shows by micrograph the effectiveness of exposure to electron beams in promoting the take of root nodules on soybean seeds;

FIG. 2 is a graph showing the percent germination of soybean seeds exposed to 200 keV electron beams; and

FIG. 3 is a graph showing the growth height at 2 weeks after planting soybean seeds exposed to 200 keV electron beams.

### DETAILED DESCRIPTION OF THE INVENTION

In the method of the present invention, plant seeds or bodies are exposed to varying energy of electron beams. Since the exposing conditions can be varied depending on the size of plant seeds and the thickness of cell waIls, the method is capable of processing seeds and plant bodies of any shape.

In the invention, low-energy electron beams are primarily used but they may be replaced by ion beams that can be controlled in penetrating power. The exposure dose is desirably in the range of 10 Gy−100 kGy. The most desirable dose, which varies with the kind of plant to be treated and the exposing conditions, is about 10 kGy. In the method of the invention, the dose of electron beams is less important than their energy which determines the degree by which the electron beams are transmitted into the plant body. If the energy of electron beams is unduly high, the degree of their penetration into the plant body becomes so high that they can damage the plant's growth capability. On the other hand, if the energy of electron beams is too low, they cannot be transmitted to the desired site. Therefore, the energy of electron beams to be applied in the invention is within the range of 10 keV to 1 MeV and the most desirable range is from 100 keV to 500 keV.

Low-energy electron beams have only small penetrating power, so in order to ensure that they are uniformly applied to the entire surface of a plan seed or body, the latter must be rotated during exposure. The effectiveness of exposure to electron beams is also exhibited if they are applied to only one surface of the target or applied to both surfaces by inverting the target after treating one surface. The simplest way of exposure is by applying electron beams to only one surface of the target and in this case it is desirable to confirm that the result is the same as is obtained by applying electron beams to both surfaces of the target or to a rotating target.

The following examples are provided for further illustrating the present invention but are in no way to be taken as limiting its scope.

The exposure dose of low-energy electron beams is highly dependent on the distance between the window of electron beams and the target. Therefore, in the following examples, the dose at the position of the target was directly measured with a film dosimeter.

### Example 1

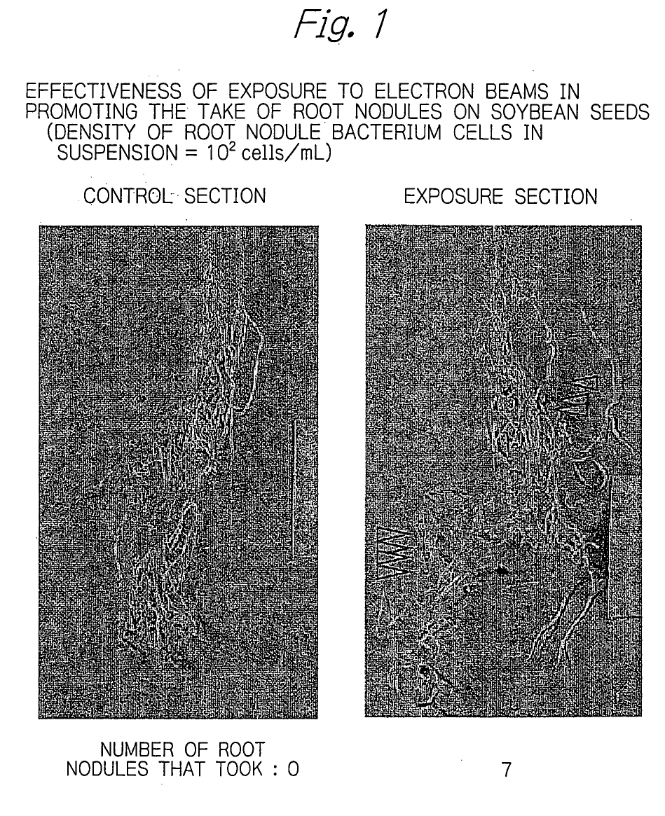

Soybean seeds were exposed on both surfaces to 10 kGy of electron beams at different energies. The seeds were then sterilized, planted and grown for 9 days. Cultured root nodule bacterium cells were diluted to a density of about 10² cells/ml to prepare a thin suspension of root nodule bacterium, in which the roots of soybean seedlings were immersed to be inoculated with the root nodule bacterium. Following 21-day cultivation, the number of root nodules that took and their total weight were calculated. The take of root nodules was hardly recognizable on the roots of soybean seedlings in the control section (which were not exposed to electron beams). On the other hand, a statistically large number of root nodules were found to take on the root of soybean seedlings in the exposure section (see Table 1 below and accompanying FIG. 1). It also became apparent that the total weight of root nodules increased significantly in the exposure section.

TABLE 1
The Number and Weight of Root Nodules That Took
on the Roots of Soybean Seedlings in the Exposure
and Control Sections (n = 5; root nodule
bacterium density = 10² cells/ml)

| | Control section | Exposure section |
|---|---|---|
| Average number | 0.6 | 5.0 |
| of takes | | |
| Total weight of | 0.01284 | 0.04502 |
| root nodules (g) | | |

### EXAMPLE 2

The procedure of Example 1 was repeated under the same conditions except that the suspension of root nodule bacterium in which the roots of soybean seedlings were immersed to be inoculated with root nodule bacterium had the density of root nodule bacterium cells increased to 10⁶ cells/ml. As a result, a lot of root nodules were found to take on the roots of soybean seedlings in both the control and exposure sections, with the take being rather higher in the exposure section (see Table 2 below). However, the difference was slight and the effectiveness of exposure to electron beams in promoting the take of root nodule bacterium was found to be more significant when the number of inocula was small.

TABLE 2
The Number and Weight of Root Nodules That Took
on the Roots of Soybean Seedlings in the Exposure
and Control Sections (n = 5; root nodule
bacterium density = 10⁶ cells/ml)

| | Control section | Exposure section |
|---|---|---|
| Average number | 22.6 | 25.8 |
| of takes | | |
| Total weight of | 0.08728 | 0.12196 |
| root nodules (g) | | |

### EXAMPLE 3

The procedure of Example 1 was repeated to expose soybean seeds to electron beams, plant and grow them for 9 days. The grown soybean seedlings were transplanted into soil and the number of root nodules that took was counted at 30 days of cultivation in a greenhouse. As it turned out, the root nodule bacterium in the soil also allowed root nodules to take in large numbers on the roots of the soybean seedlings grown in the exposure section (see Table 3 below). Compared to the take of root nodule bacterium in the control section, the take in the exposure section was almost 10-fold, clearly demonstrating the significant effectiveness of exposure to electron beams in promoting the take of root nodule bacterium.

TABLE 3
The Number and Weight of Root Nodules That Took on
the Roots of Soybean Seedlings in the Exposure and
Control Sections (cultured in soil for 30 days)

| | Control section | Exposure section |
|---|---|---|
| Number of takes | 12 | 194 |
| | 23 | 218 |

### EXAMPLE 4

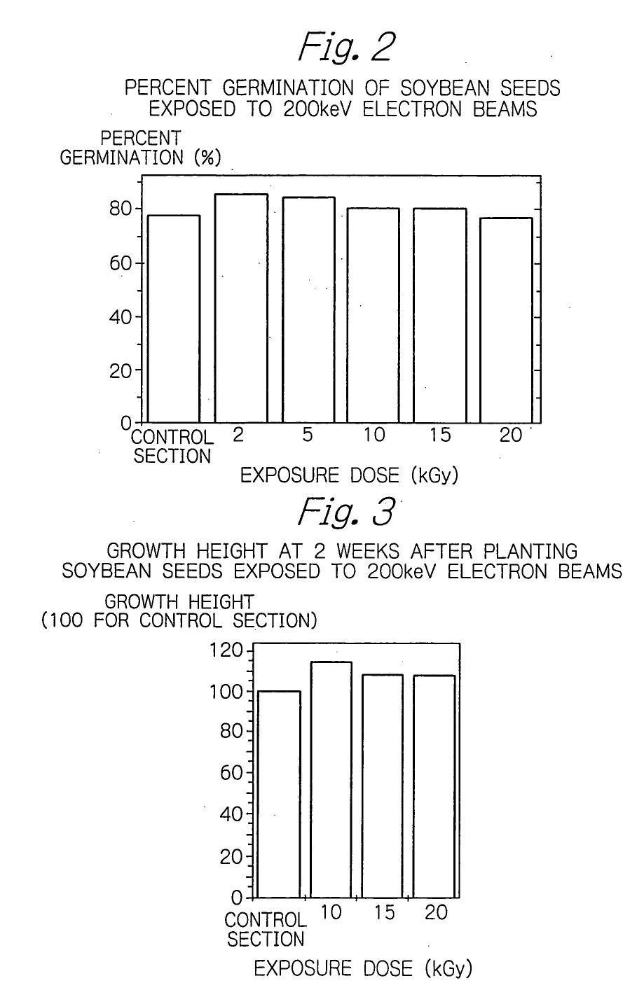

Soybean seeds on water-impregnated filter paper were irradiated with 200 keV electron beams at doses up to 20 kGy and let to grow for 2 days. As shown in FIG. 2, the percent germination increased slightly at doses of 2-15 kGy. A slight decrease was observed at the dose of 20 kGy but the difference was not significant. It is therefore concluded that with exposure to electron beams at doses of about 10 kGy which allowed the highest take of root nodule bacterium in the present invention, germination of plant seeds is not impaired but rather has the potential to be promoted.

### EXAMPLE 5

As in Example 4, soybean seeds were irradiated with 200 keV electron beams at doses up to 20 kGy and let to grow on vermiculite for 2 weeks. As FIG. 3 shows, the growth height of soybean seedlings increased at each test dose compared to the value in the control section. It was therefore clear that exposure to electron beams promoted rather than inhibited the growth of soybean seeds.

As will be understood from the foregoing, the take of root nodule bacteria can be enhanced efficiently under natural conditions by employing the promoting method of the invention. In Examples 1 and 2, soybean seeds were sterilized before planting in order to make comparison between the control section and the exposure section but in fact the irradiated seeds were surface sterilized and needed no sterilization with chemicals. This shows that the effectiveness of the present invention in promoting the take of root nodule bacteria is not due to the elimination of competing microorganisms by the sterilizing effect of exposure to electron beams but that a new mechanism worked on account of a change in the plant body itself in order to provide a technique for enhancing the interaction between the plant body and the microorganism that will take on it.

Generally speaking, the yield of soybeans is largely dependent on nitrogen fixation in symbiosis with root nodule bacteria and if it is low, a major cause is insufficient utilization of the activity of root nodules. Root nodules are formed by infection of the roots of soybean with root nodule bacteria in the soil. In fields having low soil density of root nodule bacteria and depending upon the conditions of weather, culture, etc., root nodules may not take well leading to lower yield. Therefore, exposure of plant seeds to electron beams in the present invention is anticipated to promote markedly the infection with root nodule bacteria in fields in a simple and economical way, thereby making great contribution to consistent and higher yield of soybean. In short, the present invention provides an entirely new technique that can enhance the efficiency and positivity of artificial inoculation with root nodule bacteria.

Is claimed is:

1. A method of enhancing the take of a nitrogen fixing microorganism by exposing it to low-energy electron beams.

2. The method according to claim 1, wherein the energy of electron beams is so controlled that an adequate dose for enhancing the take of the nitrogen fixing microorganism on the root of a plant is applied to the desired site on the epidermis of the plant's tissue.

3. The method according to claim 1, wherein the electron beams to be applied have an energy between 100 keV and 500 keV.

4. The method according to claim 1, wherein the electron beams are applied at a dose between 10 Gy and 100 kGy.
